(19) United States
(12) Patent Application Publication
(10) Pub. No.: US 2008/0004625 A1
(43) Pub. Date: Jan. 3, 2008

(54) BONE ANCHOR ASSEMBLIES

(76) Inventors: Thomas J. Runco, Canton, MA (US); Nicholas Miller, Taunton, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/768,350

(22) Filed: Jun. 26, 2007

Related U.S. Application Data
(60) Provisional application No. 60/805,879, filed on Jun. 27, 2006.

(51) Int. Cl. A61B 17/58

(52) U.S. Cl. 606/73

(57) ABSTRACT

Bottom-loading, for assembly, bone anchor assemblies for fixing a spinal connection element to bone and methods of assembly are described, which are particularly suited for, but not limited to, large diameter bone screws. The assembly includes a receiver member for receiving the spinal connection element, a bone-engaging shank for engaging bone and a retaining member for retaining the head of the shank within the receiver member.

### CROSS REFERENCE TO RELATED APPLICATIONS

The present application claims priority to U.S. Provisional Patent Application No. 60/805,879, filed Jun. 27, 2006, which incorporated herein by reference.

### BACKGROUND

Spinal connection systems may be used in orthopedic surgery to align and/or fix a desired relationship between adjacent vertebrae. Such systems typically include a spinal connection element, such as a relatively rigid fixation rod or plate or a dynamic connector, that is coupled to adjacent vertebrae by attaching the element to various anchoring devices, such as hooks, bolts, wires, or screws. The spinal connection element can have a predetermined contour that has been designed according to the properties of the target implantation site, and once installed, the spinal connection element holds the vertebrae in a desired spatial relationship, either until desired healing or spinal fusion has taken place, or for some longer period of time.

Spinal connection elements can be anchored to specific portions of the vertebra. Since each vertebra varies in shape and size, a variety of anchoring devices have been developed to facilitate engagement of a particular portion of the bone. Pedicle screw assemblies, for example, have a shape and size that is configured to engage pedicle bone. Such screws typically include a threaded shank that is adapted to be threaded into a vertebra, and a head portion having a spinal connection element receiving portion, which, in spinal rod applications, is usually in the form of a U-shaped slot formed in the head for receiving the connection element. A set-screw, plug, cap or similar type of closure mechanism, may be used to lock the connection element into the connection element receiving portion of the pedicle screw. In use, the shank portion of each screw may be threaded into a vertebra, and once properly positioned, a connection element may be seated through the spinal connection element portion of each screw and the connection element is locked in place by tightening a cap or similar type of closure mechanism to securely interconnect each screw and the connection element. Other anchoring devices also include hooks and other types of bone screws.

In certain procedures, such as those in the lumbar or sacral spine, it may be necessary to use a larger diameter pedicle screw capable of carrying large loads or engaging large pedicles. A difficulty in using a larger diameter screw comes from the corresponding increase in the size of the receiver head to accommodate the larger diameter screw shank, since the shank is usually assembled from the top through the opening at the proximal end of the receiver head. The increased size of the receiver head can interfere with the bony anatomy and can limit the polyaxial range of motion of the screw head. Another problem associated with manufacturing large diameter top-loading screws is the opening in the receiver head has to be larger to accept the larger diameter screw shank, which creates the need for a larger closure mechanism. It is desirable to maintain the same size opening in the receiver head such that the same size closure mechanism can be used. Accordingly, a larger diameter polyaxial screw is needed which is not top-loading.

### SUMMARY

Disclosed herein are embodiments of a bottom-loading bone anchor assembly particular suited, but not limited to, large diameter bone engaging screw shanks. In one embodiment, a bone anchor assembly for engagement to a connection element includes a receiver member having an opening at the proximal end for receiving the connection element and a bore; a bone-engaging shank having a head at a proximal end, the head sized to fit through the bore of the receiver member; and a retaining member having an inner surface shaped to accommodate the head of the shank, and an outer surface adapted to engage a seat portion of the receiver member, the retaining member retains the head of the shank within the receiver member.

A method for assembling a large diameter bottom-loading bone anchor is also disclosed. The method includes positioning an incompressible retaining member around a bone-engaging shank; inserting the bone-engaging shank having a head proximally through a bore of a receiver member having an opening for receiving a spinal connection element; and advancing the retaining member into position within a seat portion of the receiver member to retain the head of the bone-engaging shank within the receiver member.

### BRIEF DESCRIPTION OF THE FIGURES

These and other features and advantages of the bone anchor assembly and methods disclosed herein will be more fully understood by reference to the following detailed description in conjunction with the attached drawings in which like reference numerals refer to like elements through the different views. The drawings illustrate principles of the bone anchor assembly and methods disclosed herein and, although not to scale, show relative dimensions.

FIG. 1A illustrates an exploded view of an exemplary bone anchor assembly.

FIG. 1B illustrates a top view of the assembled bone anchor assembly shown in FIG. 1A.

FIG. 1C illustrates a cross-section of the assembled bone anchor assembly shown in FIG. 1A.

FIG. 1D illustrates a side view of the bone anchor assembly shown in FIG. 1C.

FIG. 1E illustrates a cross-section of an alternate embodiment of a bone anchor assembly.

FIG. 2A illustrates a perspective view of the retaining member of the bone anchor assembly shown in FIG. 1A.

FIG. 2B illustrates a cross-section view of the retaining member of the bone anchor assembly shown in FIG. 2A.

FIG. 2C illustrates a bottom isometric view of the retaining member shown in FIG. 2A.

FIG. 2D illustrates a top isometric view of an alternate embodiment of a retaining member.

FIG. 2E illustrates a cross-section view of the alternate embodiment of the retaining member shown in FIG. 2D.

FIG. 3A illustrates a perspective view of the receiver member of the bone anchor assembly shown in FIG. 1A.

FIG. 3B illustrates a cross-section view of the receiver member shown in FIG. 3A.

FIG. 3C illustrates a perspective view of an alternate embodiment of a receiver member.

FIG. 3D illustrates a cross-section view of the alternate embodiment of the receiver member shown in FIG. 3C.

FIG. 4A illustrates a perspective view of the bone-engaging shank of the bone anchor assembly shown in FIG. 1A.

FIG. 4B illustrates a cross-section of the bone-engaging shank shown in FIG. 4A.

FIG. 5A illustrates a perspective view of the compression member of the bone anchor assembly shown in FIG. 1A.

FIG. 5B illustrates a side view of the compression member shown in FIG. 5A.

FIG. 5C illustrates a cross-section view of the compression member shown in FIG. 5A.

### DETAIL DESCRIPTION OF EXEMPLARY EMBODIMENTS

Certain exemplary embodiments will now be described to provide an overall understanding of the principles of the structure, function, manufacture, and use of the bone anchor assemblies and methods disclosed herein. One or more examples of these embodiments are illustrated in the accompanying drawings. Those of ordinary skill in the art will understand that the bone anchor assemblies and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The articles “a” and “an” are used herein to refer to one or to more than one (i.e. to at least one) of the grammatical object of the article. By way of example, “an element” means one element or more than one element.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

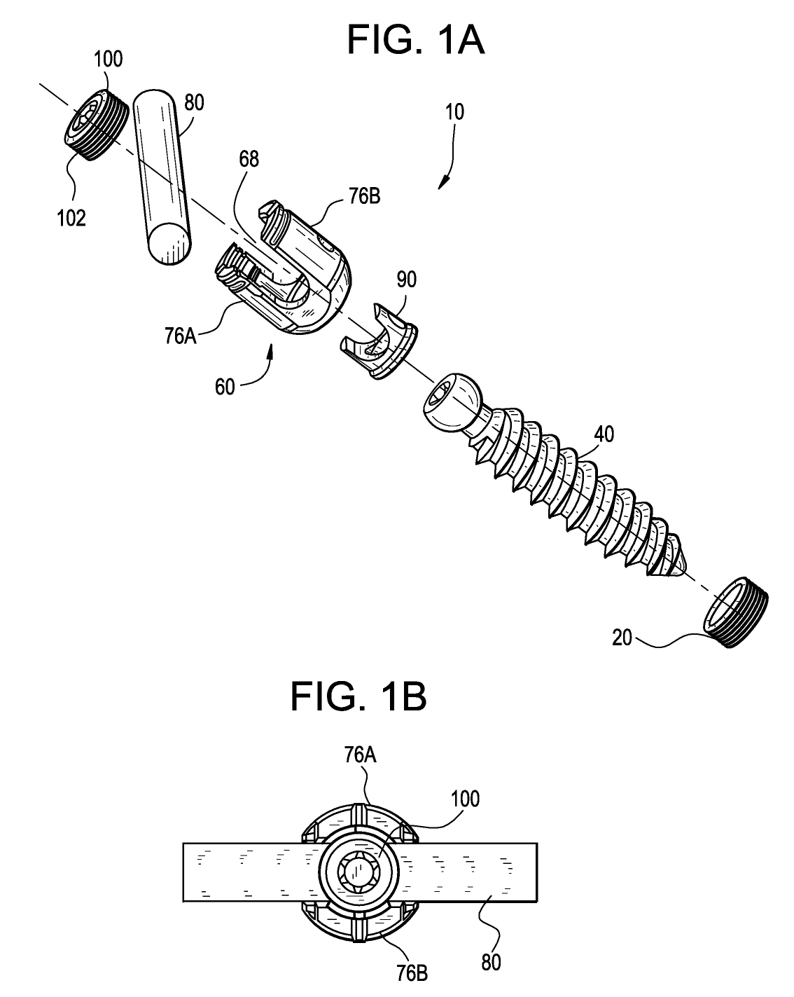

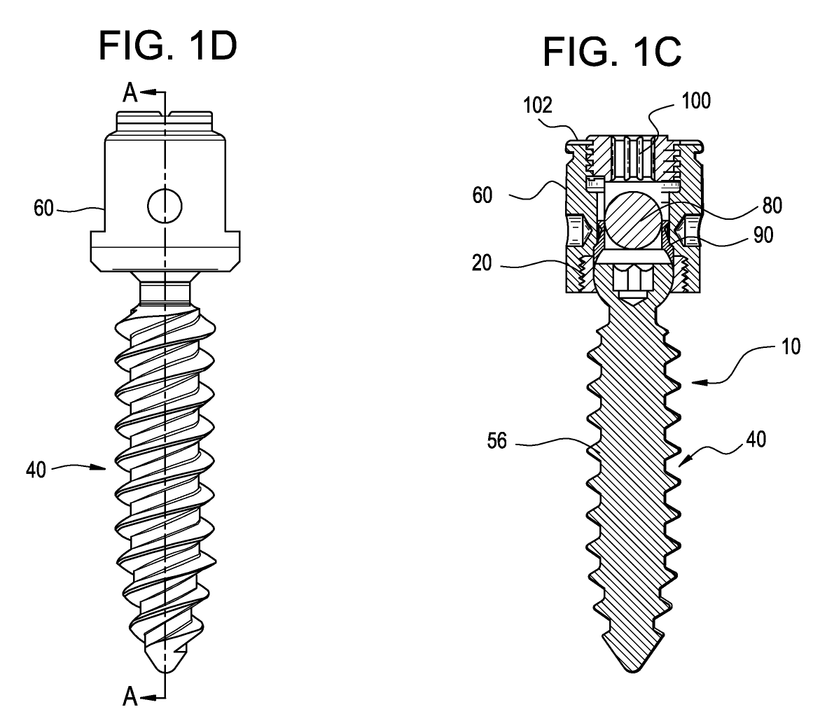

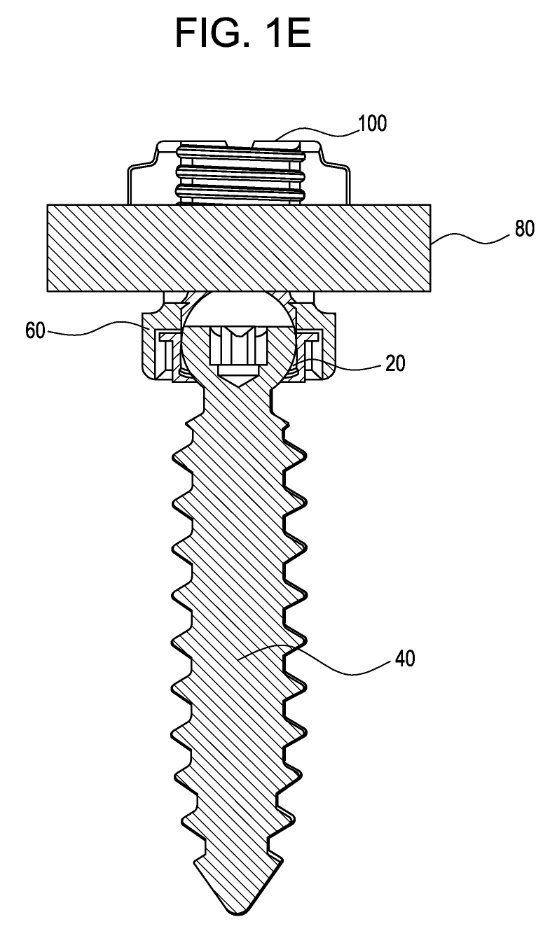

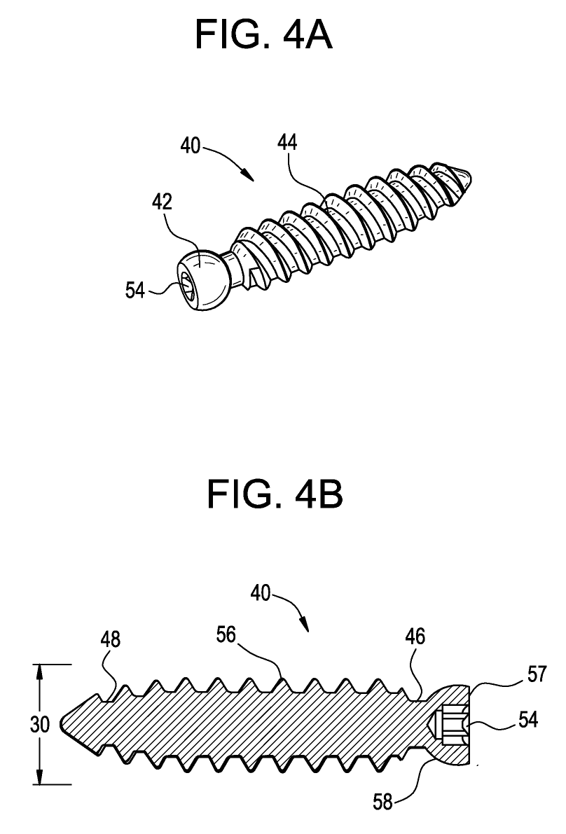

FIGS. 1-5 illustrate an exemplary embodiment of a bottom-loading bone anchor assembly. The exemplary bone anchor assembly 10 may be employed to engage one or more spinal connection elements to bone. For example, bone anchor assembly 10 may be employed to connect a spinal plate, rod (rigid or dynamic), and/or cable to a vertebra of the spine. The illustrated construct includes an exemplary spinal rod and closure mechanism in the form of a set screw. Although the exemplary bone anchor assembly 10 described below is designed primarily for use in spinal applications, one skilled in the art will appreciate that the structure, features, and principles of the exemplary bone anchor assembly 10, as well as the other exemplary embodiments described below, may be employed to couple any type of orthopedic implant to any type of bone or tissue. Non-limiting examples of applications of the bone connection anchor assembly 10 described herein include long bone fracture fixation/stabilization, small bone stabilization, lumbar spine as well as thoracic stabilization/fusion, cervical spine compression/fixation, and dynamic, non-fusion applications including facet replacement and dynamic posterior systems as well as skull fracture/reconstruction plating.

The illustrated exemplary bone anchor assembly 10 includes a bone-engaging shank 40 configured for engaging bone, a receiver member 60 for receiving a spinal connection element, and a retaining member 20 for retaining the shank 40 within the receiver member 60. The bone-engaging shank 40 extends from a proximal end 46 to a distal end 48 along a longitudinal axis. An outer surface 44 of the bone-engaging shank 40 extends between the proximal end 46 and the distal end 48. The outer surface 44 of the bone-engaging shank 40 may include one or more bone engagement mechanisms to facilitate gripping engagement of the bone anchor assembly 10 to bone. In the illustrated exemplary embodiment, for example, the bone-engaging shank 40 includes an external thread 56. The external thread 56 may extend along at least a portion of the bone-engaging shank 40. For example, in the illustrated exemplary embodiment, the external thread 56 extends from the distal end 48 to the proximal end 46 of the bone-engaging shank 40. One skilled in the art will appreciate that bone engagement mechanisms other than external thread 56 may be employed, including, for example, one or more annular ridges, multiple threads, dual lead threads, variable pitched threads, and/or any other conventional bone engagement mechanism. In the illustrated exemplary embodiment, the shank diameter 30 of bone-engaging shank 40 may be defined by the major diameter of external thread 56.

The proximal end 46 of the exemplary bone-engaging shank 40 has a head 42 configured to fit within the receiver member 60 and to facilitate adjustment of the shank 40 relative to the receiver member 60. For example, the head 42 may be generally spherical in shape to permit pivoting of the bone-engaging shank 40 relative to the receiver member 60. In the illustrated exemplary embodiment, for example, the head 42 may be in the shape of a truncated sphere having a generally planar proximal surface 57 and a generally hemispherically shaped distal surface 58. The head 42 of the shank 40 may have surface texturing, knurling, and/or ridges. A drive feature 54 may be located internally or externally on the head 42 of the shank 40.

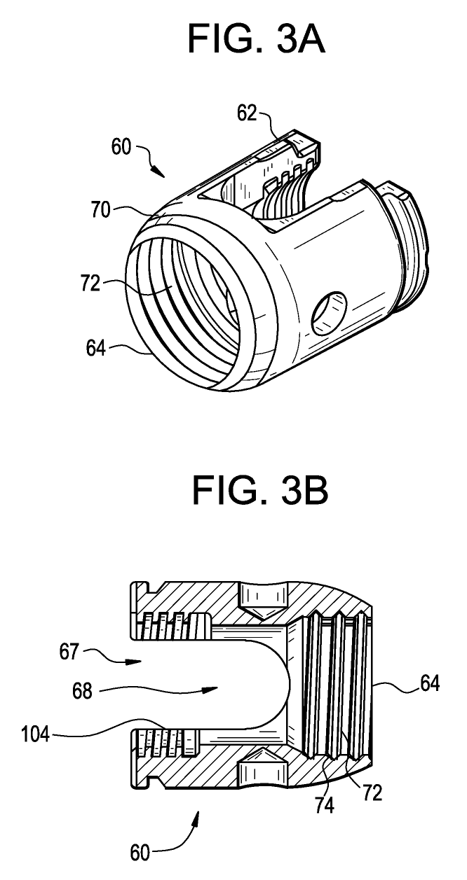

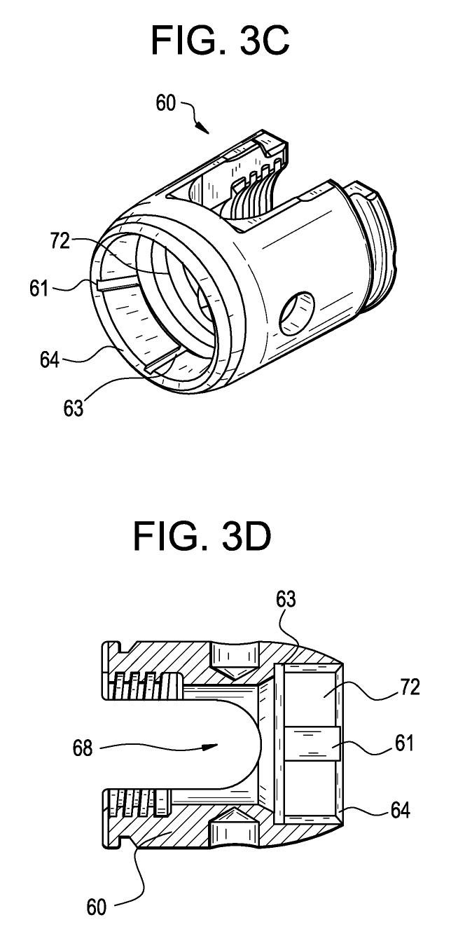

Referring to FIGS. 1A, 3A, and 3B, the receiver member 60 of the exemplary bone anchor assembly 10 includes a proximal end 62 having a cylindrical opening 67 leading to recess 68, and a distal end 70 having a bore 64 forming seat portion 72. The receiver member 60, in certain exemplary embodiments, may be configured to receive a spinal connection element and couple the spinal connection element to the bone anchor assembly. In the exemplary embodiment, for example, the recess 68 of the receiver member 60 may be sized and shaped to receive a spinal rod 80, as illustrated in FIG. 1C. For example, the receiver member 60 has a generally U-shaped cross-section defined by two legs 76A and 76B separated by recess 68. Each leg 76A, 76B is free at the proximal end 62 of the receiver member 60. In the exemplary embodiment, for example, the inner surfaces of the legs 76A, 76B have threads 104 to mate with a corresponding thread on the closure mechanism shown as a setscrew. The exemplary spinal rod 80 may be seated within the recess 68 by aligning the spinal rod 80 and the recess 68, and advancing the spinal rod 80 between the legs 76A, 76B into the recess 68. The configuration of recess 68 of the receiver member 60 may be varied to accommodate the type, size and shape of spinal connection element employed.

In the exemplary embodiment, the bore 64 of the receiver member 60 forms a seat portion 72 within the receiver member 60 to receive the retaining member 20 and, within the retaining member 20, a portion of the bone anchor assembly 10, such as the head 42 of the shank 40. The bore 64 is sized to allow at least a portion of a bone anchor assembly, such as the head 42 of the shank 40 to pass through to the seat portion 72. For example, the head 42 of the shank 40 may be inserted in the proximal direction through the bore 64 of the receiver member 60, as illustrated in FIG. 1A. The diameter of the bore 64 is greater than the diameter of the opening 67 at the proximal end 62 of the receiver member 60 between the legs 76A and 76B. The seat portion 72 may have a diameter greater than or equal to the bore 64. In the illustrated exemplary embodiment, the seat portion 72 may have threads 74 or other connection mechanisms to receive and retain the retaining member 20 within the seat portion 72. The seat portion 72 may be cylindrical, spherical, or tapered in shape or may have other shapes suitable receive and retain therein the retaining member 20 and, within the retaining member 20, a portion of the bone anchor assembly 10, such as the head 42 of the shank 40.

Retaining member 20 is sized and configured to retain the head 42 of the shank 40 within the seat portion 72 of the receiver member 60. Referring to FIG. 1C, retaining member 20 of the bone anchor assembly 10 is positionable within the seat portion 72 of the receiver member 60. The retaining member 20 may have a generally circular shape in cross section. In the exemplary embodiment, the retaining member 20 is a ring and is incompressible. The retaining member 20 forms opening 21 extending from a proximal end 12 to a distal end 14. Insertion features such as posts 15 may extend from retaining member 20 to cooperate with an insertion instrument (not shown) to place the retaining member 20 within the receiver member 60. The posts 13 may extend from either the proximal end 12 or the distal end 14. One skilled in the art will recognize that other insertion features may be used.

The retaining member 20 has an inner surface 26 and an outer surface 28. Inner surface 26 for receiving a portion of the bone achor assembly 10, such as the head 42 of the shank 40 and outer surface 28 may be adapted for engaging the seat portion 72 of the receiving member 60 to retain the retaining member 20 and, for example, the head 42 of the shank 40 within the retaining member 20 and, thus, the receiver member 60. In the exemplary embodiment, the bone anchor assembly 10 is a polyaxial bone anchor assembly. The bone-engaging shank 40 when assembled within the receiver member 60 may be pivoted to one or more angles relative to the receiver member 60. To facilitate this, the inner surface 26 may be spherical, conical, tapered, or may have other shapes suitable to permit the head 42 of the shank 40 to pivot relative to the retaining member 20, and thus the receiver member 60, in the manner of a ball and socket joint. In the illustrated exemplary embodiment, for example, the inner surface 26 may have a generally cylindrical shaped portion 27 adjacent a generally spherically shaped portion 29 having a curvature analogous to the distal surface 58 of the head 42 of the shank 40. The complementary curvatures of the head 42 and the retaining member 20 allowing pivoting between the head 42 of the shank 40 and the receiver member 60.

The retaining member 20 may have threads 25 along a portion of the inner surface 26 to aid in insertion of the retaining member 20 over the shank 40 of the bone anchor. The threads 25 may have the same thread form as the threads 56 of the shank 40 of the bone anchor. In one embodiment, the threads 25 may be positioned along the spherical shaped portion 29 of the inner surface 26 of the retaining member 20. In the case of a large diameter bone anchor assembly, the major diameter 30 of the shank 40 may be greater than the opening 21 formed by the retaining member 20 and the head 42 of the shank 40 may be greater than or equal to the opening 21 formed by the retaining member 20. In other embodiments, the major diameter 30 of the shank 40 may be less than or equal to the opening 21 formed by the retaining member 20 and the head 42 of the shank 40 may be less than to the opening 21 formed by the retaining member 20

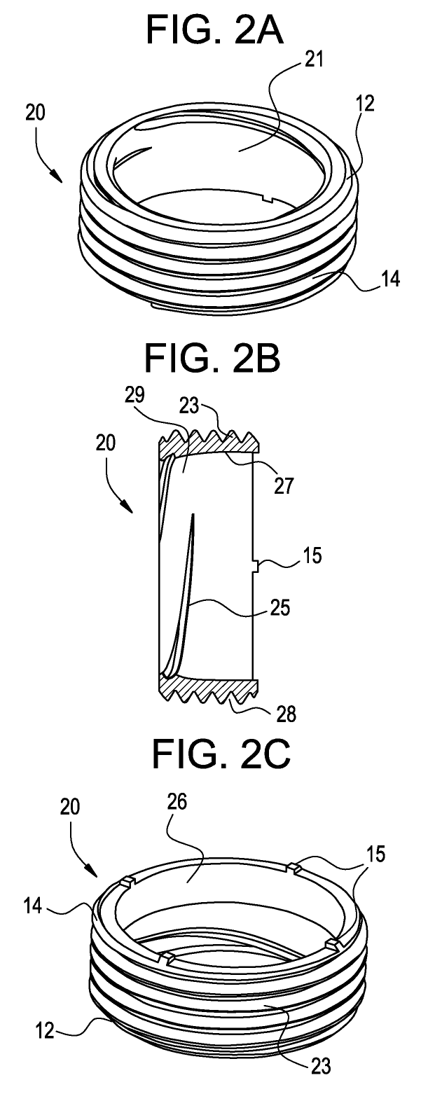

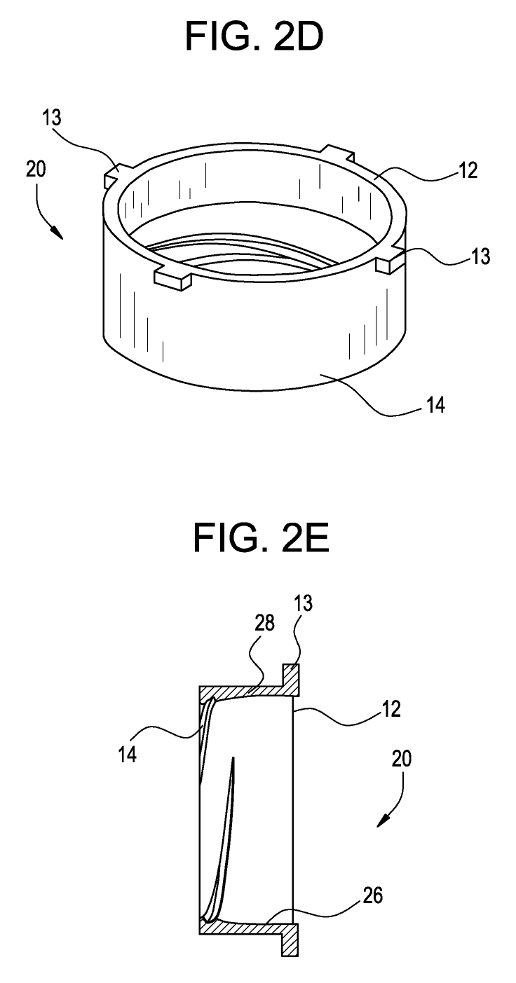

In one embodiment, the outer surface 28 of the retaining member 20 may have threads 23 to engage the threads 74 of the seat portion 72 of the receiver member 60 as illustrated in FIG. 1C. Alternately, the retaining member 20 may have tabs 13 as shown in FIG. 2D extending from the outer surface 28 at the proximal end 12 to engage a channel 61 extending along the bore 64 and the seat portion 72 of the receiver member 60. The channel 61 may be in the form of a J-slot such that the tab 13 of the retaining member 20 slides along the channel 61 and is rotated at the proximal end 63 of the channel 61 to hold the retaining member 20 in position within the seat portion 72 of the receiver member 60. The channel 61 of the alternate embodiment of the receiver member 60 is illustrated in FIGS. 3C and 3D. A cross-section of the bone anchor assembly with the alternate embodiment of the retaining member is shown in FIG. 1E. In other exemplary embodiments, the retaining member 20 may be tapered or have any other shape that allows assembly within the seat portion 72 of the receiver member 60.

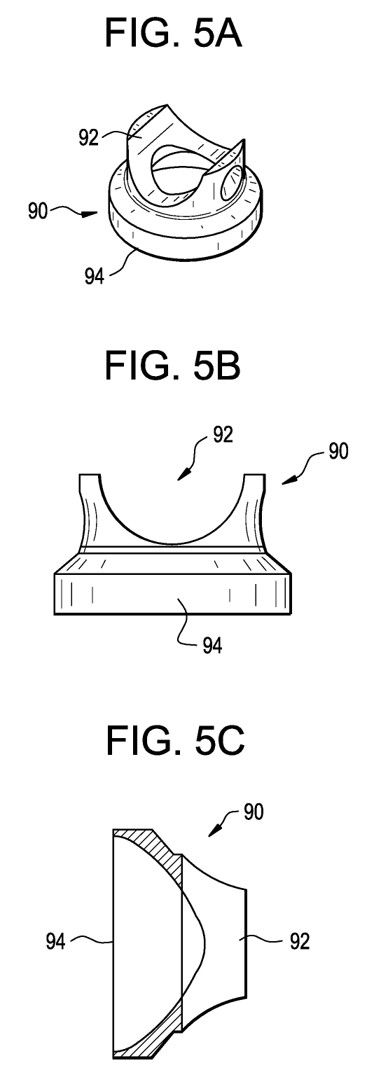

The bone anchor assembly 10 may optionally include a compression member 90 as shown in FIGS. 5A-C positionable within the receiver member 60 between the spinal connection element and the bone anchor. As illustrated in FIG. 1C, the compression member 90 may be positioned within the recess 68 between the spinal rod 80 and the head 42 of the shank 40. In the exemplary embodiment, the compression member 90 may have a proximal first surface 92 for engaging the spinal connection element and an opposing distal second surface 94 for engaging the head 42 of the shank 40.

The exemplary bone anchor assembly 10 may include a closure mechanism 100 that secures the spinal connection element to the bone anchor assembly. Referring to FIG. 1A, the closure mechanism 100 secures the exemplary spinal rod 80 within the recess 68 of the receiver member 60. The closure mechanism 100 may engage the proximal end 62 of the receiver member 60 or, in other exemplary embodiments, may engage other portion(s) of the receiver member 60. The exemplary closure mechanism 100 is an internal setscrew that engages an inner surface of the proximal end 62 of the receiver member 60. For example, the closure mechanism 100 may have external threads 102 that engage internal threads 104 provided on the proximal end 62 of the receiving member 60. Distal advancement of the closure mechanism 100 into engagement of the spinal rod 80, seats the spinal rod 80 in the proximal surface 92 of the compression member 90. The compression member 90 then is advanced onto the head 42 of the bone-engaging shank 40 thereby fixing the relative movement of the head 42 in relation to the receiver member 60. In the case of large diameter bone anchor assemblies, the major diameter of the bone-engaging shank 30 may be greater than the diameter of the closure mechanism 100.

One skilled in the art will appreciate that other types of closure mechanisms may be employed. For example, an external closure mechanism positionable around the outer surface of the legs 76A, 76B of the receiving member 60 may be employed. In other exemplary embodiments, the closure mechanism may comprise an external and an internal closure mechanism, a non-threaded twist-in cap, and/or any other conventional closure mechanism.

The components of the bone anchor assembly may be manufactured from any biocompatible material, including, for example, metals and metal alloys such as titanium and stainless steel, polymers, and/or ceramics. The components may be manufactured of the same or different materials. In one exemplary method of manufacturing, the bone-engaging shank 40, the retaining member 20 and the receiver member 60 are separately constructed and assembled prior to implantation. The retaining member 20 is advanced around the threads 56 of the bone-engaging shank 40, the major diameter of the shank being greater than the opening 21 of the retaining member 20. The retaining member 20 and shank 40 are proximally inserted through the bore 64 of the receiver member 60. The retaining member 20 is positioned within the seat portion 72 of the receiver member to retain the head 42 of the bone-engaging shank 40 within the receiver member 60.

In one embodiment, the retaining member 20 may be threadedly inserted over the threads 56 of the shank 40 until it reaches the head 42 of the shank 40. The threads 25 of the retaining member 20 mate with threads 56 of the shank 40 to advance the retaining ring 20 into position. The shank 40 and retaining member 20 are inserted proximally through the bore 64 of the receiver member 60. The retaining member 20 is advanced around the head 42 until it is seated within the seat portion 72 of the receiver member 60. The head 42 is captured within the spherically shaped portion 29 of the retaining member 20. If the retaining member 20 is threaded into position within the seat portion 72 after positioning, the threads may be deformed or staked to provide additional retention of the retaining member 20 within the receiver member 60. Alternately, the retaining member 20 may be welded, swaged or staked in position within the receiver member 60. If the retaining member 20 has tabs 13, the tabs 13 are aligned with the channel 61 of the receiver member and advanced to the proximal end of the channel 61 and rotated to retain the retaining member 20 and the shank 40 within the receiver member 60.

While the bone anchor assembly and methods of the present invention have been particularly shown and described with reference to the exemplary embodiments thereof, those of ordinary skill in the art will understand that various changes may be made in the form and details herein without departing from the spirit and scope of the present invention. Those of ordinary skill in the art will recognize or be able to ascertain many equivalents to the exemplary embodiments described specifically herein by using no more than routine experimentation. Such equivalents are intended to be encompassed by the scope of the present invention and the appended claims.

What is claimed:

1. A bone anchor assembly for engagement to a connection element comprising:
a receiver member having an opening at the proximal end for receiving the connection element;
a shank having a head at a proximal end and an external thread having a major diameter for engaging bone at the distal end; and
a retaining member having an opening extending from a proximal end to a distal end, an inner surface shaped to accommodate the head of the shank, and an outer surface adapted to engage the receiver member, the major diameter of the shank being greater than the opening formed by the retaining member.

2. The bone anchor assembly of claim 1 wherein the head of the shank has a generally spherical shape.

3. The bone anchor assembly of claim 2 wherein the inner surface of the retaining member has a generally spherical shaped portion to accommodate the head of the shank.

4. The bone anchor assembly of claim 1 wherein the outer surface of the retaining member is threaded.

5. The bone anchor assembly of claim 4 wherein the receiver member has a threaded seat portion.

6. The bone anchor assembly of claim 1 wherein the outer surface of the retaining member has a tab for engaging a bore of the receiver member.

7. The bone anchor assembly of claim 6 wherein the bore of the receiver member has a channel extending proximally along the longitudinal axis of the receiver member.

8. The bone anchor assembly of claim 7 wherein the channel has a J-slot configuration for engaging the tab of the retaining member.

9. The bone anchor assembly of claim 1 further comprising a closure mechanism.

10. The bone anchor assembly of claim 9 wherein the major diameter of the shank is greater than the major diameter of the closure mechanism.

11. The bone anchor assembly of claim 1 wherein the major diameter of the shank is greater than the opening of the receiver member.

12. The bone anchor assembly of claim 1 wherein the retaining member has a generally circular shape.

13. The bone anchor assembly of claim 1, wherein a portion of the inner surface of the retaining member has threads.

14. The bone anchor assembly of claim 1, wherein the threads on the inner surface have the same thread form as the threads on the shank.

15. The bone anchor assembly of claim 1, wherein the retaining member is incompressible.

16. The bone anchor assembly of claim 1 further comprising a compression member having a proximal surface for engaging the spinal connection element and a distal surface for engaging the head of the bone-engaging shank.

17. A method of assembly of a bone anchor assembly comprising:
advancing a retaining member having an opening around the threads of a bone-engaging shank, the major diameter of the shank being greater than the opening;
proximally inserting the retaining member and shank through a bore of a receiver member having an opening for receiving a spinal connection element; and
positioning the retaining member within a seat portion of the receiver member to retain the head of the bone-engaging shank within the receiver member.

18. The method of claim 17 wherein the retaining member is threadedly advanced around the threads of the bone-engaging shank.

19. The method of claim 17 wherein the retaining member is positioned within the seat portion by threading.

20. The method of claim 17 wherein tabs of the retaining member are aligned and inserted along a channel within the receiver member.

21. The method of claim 17 further comprising:
swaging, welding or staking of the retaining member to the receiver member.

22. A bone anchor assembly comprising:
a receiver member having an opening at the proximal end thereof configured to seat a spinal connection element therein and a distal opening having internal threads formed therein;
a shank having a proximal end and distal end configured to engage bone;
a retaining member having an opening extending from a proximal end to a distal end, an inner surface shaped to receive and retain the proximal end to permit pivoting of the shank relative to the retaining member and the receiver member, and an outer surface having external threads for engaging the internal threads of the distal opening of the receiver member; and
a closure mechanism configured to connect to the proximal end of the receiver member and retain the spinal connection element relative to the receiver member.
